(19) United States
(12) Patent Application Publication
(10) Pub. No.: US 2023/0201660 A1
(43) Pub. Date: Jun. 29, 2023

(54) MEDICAL DEVICE RADIO FREQUENCY TRANSCEIVER AND METHODS OF USE THEREOF

(71) Applicant: Renovia Inc., Boston, MA (US)

(72) Inventors: Jose BOHORQUEZ, Orlando, FL (US); Mohamad FOUSTOK, Parkland, FL (US); Nathan WEIBLEY, Chicago, IL (US)

(21) Appl. No.: 17/926,192

(22) PCT Filed: May 19, 2021
(86) PCT No.: PCT/US2021/033155

§ 371 Date: Nov. 18, 2022

Related U.S. Application Data
(60) Provisional application No. 63/027,162, filed on May 19, 2020.

(51) Int. Cl. A63B 23/20; A61B 5/22

(52) U.S. Cl. CPC A63B 23/20; A61B 5/224; A61H 2201/5097

(57) ABSTRACT

Described herein are radio frequency (RF) transceivers that act as a relay device to provide communication between a sensor of a medical device (e.g., a medical device implanted within the body) and a peripheral device, such as a smartphone. The medical device may be an intraurethral, intrarectal, or intravaginal device. The sensor of the medical device may be a microelectromechanical (MEM) sensor, such as an accelerometer.

### BACKGROUND OF THE INVENTION

Sensors, such as sensors of a medical device, typically need to communicate wirelessly with peripheral devices in order to transmit data, e.g., in real time. Peripheral devices, such as a smartphone or tablet, represent a widely available interface that is used by subjects, e.g., patients, to effectively communicate with the sensor(s) of a medical device. In many instances, signals from a medical device inserted into or implanted within the body of a subject have to pass through the body tissue of the subject in order to communicate with a peripheral device.

Smartphones are typically equipped with a Bluetooth Low Energy (BLE)-enabled device that is configured to transmit at a specific radio frequency (RF). Transmission of a BLE signal through the human body can result in severe attenuation of the signal (e.g., transmission of a BLE signal through only a few inches of body tissue results in almost total attenuation at safe power levels). Although transmission of the BLE signal at a higher power level could be used to overcome this limitation, the higher power level could expose the subject to dangerous levels of energy. Furthermore, the attenuation of an RF signal is typically proportional to the frequency, and higher frequencies exhibit higher attenuation.

Accordingly, new devices, systems, and methods are needed to overcome signal attenuation in connection with medical devices that operate by transmitting data signals from internal medical sensors to an external peripheral device, such as a smartphone.

### SUMMARY OF THE INVENTION

In one aspect, featured is a radio frequency (RF) transceiver configured to transmit and receive a first RF signal at a first frequency of greater than about 1 GHz and a second RF signal at a second frequency of less than about 1 GHz. The first frequency may be greater than about 2 GHz (e.g., from about 2 GHz to about 3 GHz, e.g., about 2.45 GHz). The second frequency may be from about 1 MHz to about 1 GHz (e.g., about 915 MHz, about 433 MHz, or about 402 MHz).

The transceiver may be configured to transmit to and to receive an RF signal from a Bluetooth Low Energy (BLE) device that is configured to transmit and/or receive the first RF signal at the first frequency, wherein, optionally, the BLE device is configured to transmit and/or receive the first RF signal at the first frequency. The transceiver may be configured to transmit to and to receive the second RF signal at the second frequency from an industrial, scientific, and medical (ISM) or Medical Implant Communications Systems (MICS) device. The ISM or MICS device may be configured to transmit and/or receive the second RF signal at the second frequency. The transceiver may further include a microcontroller.

In another aspect, featured is a system that includes a relay device including the transceiver of any of the above embodiments, a first device that includes a first transmitter and/or receiver configured to transmit and/or receive the first RF signal at the first frequency; and a second device that includes a second transmitter and/or receiver configured to transmit and/or receive the second RF signal at the second frequency. The first device may be a BLE device. The second device may be an ISM or MICS RF device.

The system may further include a peripheral device. The peripheral device may include the first device and/or the relay device. The peripheral device may be, for example, a smartphone, tablet, computer, or smart watch.

The system may include a medical device. The medical device may include the second device. The system may include one or more microcontrollers, e.g., that are in the medical device and/or the peripheral device. At least a portion of the medical device is configured to be inserted or implanted inside a body or body cavity of a subject (e.g., human subject). For example, the portion of the medical device that is inserted or implanted may include the second device. The medical device may be an intravaginal, intrarectal, or intraurethral device.

The medical device may include at least one sensor, e.g., that includes, communicates with, or is connected to (e.g., via a wired or wireless connection), the second device. The at least one sensor may be configured to transmit a signal to the second device. The medical device may include a plurality of sensors, e.g., that are positioned along a length of the medical device. The at least one sensor may be a position or movement sensor, e.g., a microelectromechanical (MEM) sensor, e.g., an accelerometer. In some embodiments, the medical device further includes at least one sensor selected from the group consisting of a pressure sensor, a flow sensor, a muscle quality sensor, a muscle strength sensor, a pH sensor, a humidity sensor, a temperature sensor, a hormone sensor, a toxin sensor, and a hall effect sensor.

In another aspect, featured is a method of using the system of any of the above embodiments. The method may include transmitting the first RF signal at the first frequency from the first device to the relay device; receiving the first RF signal at the first frequency by the relay device; converting the first RF signal at the first frequency by the relay device to the second RF signal at the second frequency; and transmitting the second RF signal at the second frequency to the second device. In another embodiment, e.g., the reverse, may include transmitting the second RF signal at the second frequency from the second device to the relay device; receiving the second RF signal at the second frequency by the relay device; converting the second RF signal at the second frequency by the relay device to the first RF signal at the first frequency; and transmitting the first RF signal at the first frequency to the first device.

In another aspect, featured is a method of detecting a pelvic floor movement with the system of any of the above embodiments. The method may include inserting the intravaginal, intrarectal, or intraurethral device into the body of the subject; obtaining a signal from the position or movement sensor upon engagement or contraction of the pelvic floor; and transmitting the signal from the position or movement sensor to the peripheral device via the transceiver. The first frequency may be greater than about 2 GHz (e.g., from about 2 GHz to about 3 GHz, e.g., about 2.45 GHz). The second frequency may be from about 1 MHz to about 1 GHz (e.g., about 915 MHz, about 433 MHz, or about 402 MHz). For example, the first frequency may be about 2.45 GHz and the second RF frequency may be about 915 MHz.

The intravaginal, intrarectal, or intraurethral device may include a plurality of position or movement sensors, e.g., MEM sensors, e.g., accelerometers. The MEM sensors ay be positioned along a length of the device. The method may produce a position of the subject’s vagina, rectum, or urethra that is generated by the plurality of position or movement sensors is displayed on a graphical user interface of the peripheral device. The position of the subject’s vagina, rectum, or urethra may be displayed on the graphical user interface prior to performance of a pelvic floor exercise and/or after performance of the pelvic floor exercise. The position of the subject’s vagina, rectum, or urethra prior to performance of a pelvic floor exercise and/or after performance of the pelvic floor exercise may also be recorded.

In another aspect, featured is a medical device (e.g., intravaginal, intrarectal, or intraurethral device) that includes the transceiver of any of the above embodiments. The medical device may include a plurality of position or movement sensors, e.g., MEM sensors, e.g., accelerometers. The MEM sensors may be positioned along a length of the device.

In another aspect, featured is a kit that includes one or more of a relay device with the transceiver of any of the above embodiments; a first device that includes a first transmitter and/or receiver configured to transmit and/or receive the first RF signal at the first frequency; and a second device that includes a second transmitter and/or receiver configured to transmit and/or receive the second RF signal at the second frequency.

The kit may further include a medical device (e.g., an intravaginal, intrarectal, or intraurethral device). The intravaginal, intrarectal, or intraurethral device may include a plurality of position or movement sensors, e.g., MEM sensors, e.g., accelerometers. The MEM sensors ay be positioned along a length of the device. The medical device may include the second device. The kit may further include instructions for use thereof.

### DEFINITIONS

As used herein, the singular form “a,” “an,” and “the” includes plural references unless indicated otherwise.

As used herein, the terms “about” and “approximately” mean +/- 10% of the recited value. For example, a frequency of about 433 MHz refers to a frequency range of from 389.7 MHz to 476.3 MHz.

As used herein, the term “in proximity to” and “proximal” refers to a location near a tissue surface (e.g., about 0.01-5 mm from, or adjacent to, the tissue surface, e.g., surrounding the cervix or vaginal cuff of a subject).

As used herein, the term “feedback” or “biofeedback” refers to information that can be used to train a subject to change physiological activity (e.g., pelvic floor muscle function) for the purpose of improving health and performance (e.g., treating, reducing, and/or preventing the occurrence of or the symptoms of a pelvic floor disorder (PFD)). Biofeedback may also include information collected by a sensor of a device, such as an intraurethral, intravaginal, or intrarectal device, during daily monitoring, e.g., in substantially real-time, while a user performs a daily activity. The information can be reviewed substantially in real-time or can be accessed for review later. Instruments, such as a medical device described herein, can be used to measure physiological activity, such as muscle activity (e.g., position or movement), pressure (e.g., bladder or vaginal pressure), muscle quality, pH (e.g., vaginal canal pH), temperature, and humidity, and to provide this information as biofeedback to the subject. A sensor of a device described herein may also be used to measure the level of a molecule, e.g., the level of a hormone and/or the level of a toxin, and to provide this information as biofeedback to the subject. The presentation of this information to the subject may be transmitted via a visual, audible, or tactile signal.

As used herein, the term “diagnosis” refers to the identification or classification of a disease or condition (e.g., a pelvic floor disorder). For example, “diagnosis” may refer to identification of a particular type of urinary incontinence.

A “disorder” is any condition that would benefit from treatment including, but not limited to, chronic and acute disorders or diseases, including those pathological conditions which predispose the subject to the disorder in question.

As used herein, the term “monitoring” refers to a use of a medical device, such as an intraurethral, intrarectal, or intravaginal device as described herein, to collect, track, and/or store data, e.g., data obtained from sensor(s) of a device described herein. The monitoring can occur, e.g., when the device is positioned within the body, such as within the vaginal cavity, rectum, or urethra of a user and/or when the device is used during a diagnostic or treatment period.

As used herein, the terms “pelvic floor lift” and “PFL” refers to a movement of the pelvic floor (e.g., the muscle fibers of the levator ani (e.g., the pubococcygeus, ileococcygeus, coccygeus, and puborectalis muscles, as well as movement of perineal muscles and anal sphincter) and the associated connective tissues which span the area in a spherical form from the pubic bone anteriorly to the sacrum posteriorly and to the adjoining bony structure joining these two bones, which is characterized by an upward movement (e.g., a lifting movement, such as a movement in the cranial direction) of the pelvic floor. The movement of the pelvic floor during a PFL is a distinctly-described component of the collective action of the entire pelvic floor (e.g., the levator ani, urethral and anal sphincters, bulbocavernosus, ischiocavernosus, superficial transverse perineal muscles) whereby the combined lifting and circumferentially-directed squeezing action is produced when all muscles are activated simultaneously. A PFL may involve the selective engagement of the levator ani component of the pelvic floor.

As used herein, the terms “pelvic floor relaxation” and “PFR” refers to a movement of the pelvic floor (e.g., the muscle fibers of the levator ani (e.g., the pubococcygeus, iliococcygeus, coccygeus, and puborectalis muscles) and the associated connective tissues which span the area in a spherical form from the pubic bone anteriorly to the sacrum posteriorly and to the adjoining bony structure joining these two bones), which is characterized by a relaxation (e.g., a downward movement, such as a movement in the caudal direction) of the pelvic floor. The movement of the pelvic floor during a PFR is distinct from the concentric contraction (e.g., shortening contraction) of the PFL, and represents the lengthening or relaxation of the muscle fibers.

As used herein, “real-time” refers to the actual time during which an event, such as a daily activity, occurs.

As used herein, “sensor data” refers to a measurement (e.g., any one or more of measurements of muscle (e.g., pelvic floor muscle) movement, muscle quality, muscle strength, pressure, and measurements of other conditions, such as pH, temperature, and/or moisture (e.g., in the vagina, urethra, or rectum)), which characterize a subject’s pelvic floor health and are obtained by a sensor(s), as described herein, of a medical device, such as an intraurethral, intrarectal, or intravaginal device described herein. Sensor data may also be collected that relate to a pelvic floor movement to, e.g., urinary or fecal incontinence or urge. These data can be used, e.g., to diagnose urinary or fecal incontinence.

As used herein, “radio frequency” refers to electromagnetic waves that have a frequency in the range from 10³ Hz to 10¹² Hz.

As used herein, the terms “subject” and “patient” may be used interchangeably to refer to a mammal, such as a human.

As used herein, the terms “reducing” and “inhibiting” are defined as the ability to cause an overall decrease of about 10%, 20%, 30%, 40%, 50%, 60%, 70%, 75%, 80%, 85%, 90%, 95%, or more in a measurable metric. Reduce or inhibit can refer, for example, to the symptoms of the pelvic floor disorder (PFD) being treated.

As used herein, the term “treating” refers to providing a therapy to a subject in need thereof (e.g., to treat or reduce the likelihood of urinary or fecal incontinence, or urge associated therewith), in particular in conjunction with the use of a device (e.g., a urodynamic catheter, intravaginal device, or intrarectal device), system, or method described herein. To “treat disease” or use for “therapeutic treatment” includes administering treatment to a subject already suffering from a disease to improve or stabilize the subject’s condition. To “prevent” or “reduce the likelihood of developing” disease refers to prophylactic treatment of a subject who is not yet ill or symptomatic, but who is susceptible to, or otherwise at risk of, a particular disease, such as a urinary or fecal incontinence or pelvic organ prolapse.

As used herein, “female urogenital system” refers to the organ system of the female reproductive system, which includes, e.g., the Bartholin’s glands, cervix, clitoris, clitoral frenulum, clitoral glans (glans clitoridis), clitoral hood, fallopian tubes, labia, labia majora, labia minora, frenulum of labia minora, ovaries, skene’s gland, uterus, vagina, and vulva; the urinary system, which includes, e.g., the kidneys, ureters, bladder, and the urethra; and the surrounding and supporting nerves and musculature.

As used herein, “male urogenital system” refers to the organ system of the male reproductive system, which includes, e.g., the bladder, pubic bone, external urethral sphincter, penis, corpus, cavernosum, glans penis, foreskin, urethral opening, sigmoid colon, rectum, seminal vesicle, ejaculatory duct, prostate gland, Cowper’s glad, anus, vas deferens, epididymis, testis, and scrotum, kidneys, ureters, bladder, and the urethra; and the surrounding and supporting nerves and musculature.

As used herein, “vaginal cuff” refers to the sutured tissue at the top of the vaginal canal remaining after removal of the cervix (e.g., during a hysterectomy).

As used herein, “urinary incontinence” refers to the leaking of urine from the bladder. Incontinence can range from leaking just a few drops of urine to complete emptying of the bladder. Urinary incontinence can be divided into three main types: stress urinary incontinence (SUI), urgency urinary incontinence, and mixed incontinence. Stress urinary incontinence is leaking urine when coughing, laughing, or sneezing. Leaks can also happen when a subject (e.g., female subject) walks, runs, or exercises. Urgency urinary incontinence is a sudden strong urge to urinate that is hard to stop. Women with this type of urinary incontinence may leak urine on the way to the bathroom. Mixed incontinence combines symptoms of both stress and urgency urinary incontinence.

As used herein, “pelvic floor” refers to the muscular area at the base of the abdomen attached to the pelvis.

As used herein, “pelvic floor disorders” or “PFDs” refers to disorders affecting the muscles and tissues that support the pelvic organs. These disorders may result in loss of control of the bladder or bowels or may cause one or more pelvic organs to drop downward, resulting in prolapse.

As used herein, “urodynamic catheter” refers to urethral catheter configured for use in performing one or more urodynamic measurements. The catheter may have multiple (e.g., 2 or 3) lumens.

### BRIEF DESCRIPTION OF THE DRAWINGS

FIG. 1 is a schematic diagram showing the topology of a system as described herein that includes a device (e.g., a medical device with a sensor implanted in a body or inserted into a body cavity, such as in the context of a medical device), a relay device, and a peripheral device, such as a smartphone. The relay device communicates with the implanted device via the 915 MHz ISM band and with the smartphone via the 2.4 GHz BLE band.

FIG. 2 is a schematic diagram showing a system as described herein that includes an intravaginal device (2) with a flexible printed circuit board (PCB) (4) that contains six sensors and a microcontroller (MCU), and a peripheral device (6), such as a smartphone. The case of the intravaginal device contains an RF transceiver (8) that facilitates communication with the MCU of the intravaginal device via the 915 MHz ISM band and with the smartphone via the 2.4 GHz BLE band. The peripheral device is also able to transmit data to a cloud-based server (10) or web-based dashboard (12) in which data from the intravaginal device can be stored and/or analyzed.

FIG. 3 is a schematic diagram showing a system that does not include a relay device. Intravaginal device (2) communicates with a peripheral device (6), such as a smartphone, via the 2.4 GHz BLE band. The peripheral device is also able to transmit data to a cloud-based server (10) or web-based dashboard (12) in which data from the intravaginal device can be stored and/or analyzed.

### DETAILED DESCRIPTION

Described herein are radio frequency (RF) transceivers that act as a relay device to facilitate communication between a sensor of a medical device (e.g., a medical device inserted into or implanted within the body or body cavity of a subject) and a peripheral device, such as a smartphone, tablet, or laptop computer. The medical device may be, for example, an intraurethral, intrarectal, or intravaginal device. The sensor of the medical device may be a microelectromechanical (MEM) sensor, such as an accelerometer.

A sensor of a medical device may be configured to communicate wirelessly with a peripheral device, e.g., via a microcontroller, in order to transmit data, e.g., in real time. When the medical device is implanted within (e.g., at least partially within) the body of a subject, the sensor may not effectively transmit its signal to the peripheral device due to signal attenuation caused by body tissue. The signal attenuation is directly proportional to frequency, and a higher frequency is subject to a higher level of attenuation. In some cases, the peripheral device may only have one or more radios that transmit and receive a signal at higher frequencies. For example, most mobile phones are equipped for wireless communication to peripheral devices through Bluetooth, Bluetooth Low Energy, or WiFi, all of which operate at frequencies of 2.4 GHz or higher. Accordingly, the present disclosure describes devices, systems, and method that solve this problem by using a radio frequency transceiver that receives a signal from a sensor within the body (e.g., via a microcontroller), at a low frequency level (e.g., less than 1 GHz) and facilitates communication of the low frequency signal to an RF transmitter and/or receiver (e.g., transceiver) configured to receive the signal at a high frequency (e.g., greater than 1 GHz), and vice versa. The RF transceiver converts and transmits the signal between the implanted device and the transmitter and/or receiver (e.g., transceiver) in the peripheral device. The devices, systems, and methods of the present disclosure are described in more detail below.

### Radio Frequency Transceiver

A radio frequency (RF) transceiver transmits and receives RF electromagnetic waves that have a frequency in the range from 10³ Hz to 10¹² Hz. An RF transceiver may be a small sized electronic device that can transmit and receive a radio signal between two devices, e.g., to communicate with another device wirelessly. While a transceiver is a single module with both transmitting and receiving capabilities, one of skill in the art would understand that the transceivers described herein also refer to a device or system that includes a separate transmitter and receiver operatively configured to perform the same or similar function as a transceiver with both capabilities.

A transceiver is a unit that contains both a transmitter and a receiver and shares common circuitry or a single housing. A module that includes a transceiver typically incorporates a printed circuit board (PCB), transmit and receive circuit, an antenna, and a serial interface for communication to a host processor. A transmitter module is a small PCB sub-assembly capable of transmitting a radio wave and modulating that wave to carry data. A transmitter module is usually implemented alongside a microcontroller, which will provide data to the module, which can be transmitted. A receiver module receives the modulated RF signal and demodulates it.

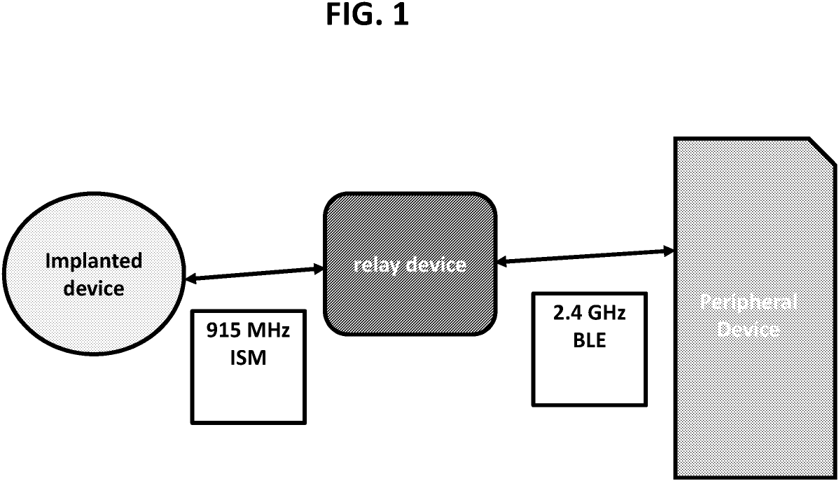

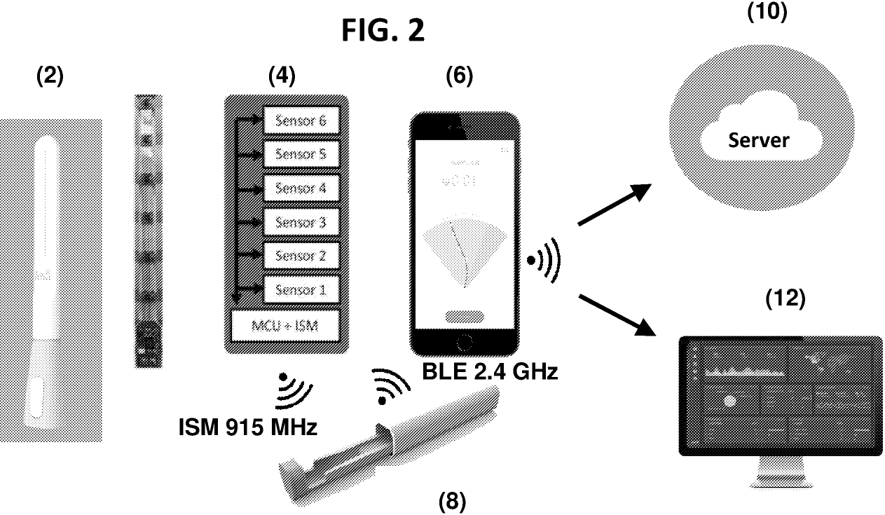

The RF transceivers described herein are configured to transmit and receive a first RF signal at a first frequency of greater than about 1 GHz and a second RF signal at a second frequency of less than about 1 GHz. For example, the first frequency may be greater than about 2 GHz, such as from about 2 GHz to about 3 GHz. In some embodiments, the first frequency may be from about 1 GHz to about 3 GHz (e.g., about 1 GHz, 1.1 GHz, 1.2 GHz, 1.3 GHz, 1.4 GHz, 1.5 GHz, 1.6 GHz, 1.7 GHz, 1.8 GHz, 1.9 GHz, 2.0 GHz, 2.1 GHz, 2.3 GHz, 2.4 GHz, 2.5 GHz, 2.6 GHz, 2.7 GHz, 2.8 GHz, 2.9 GHz, 3.0 GHz. In some embodiments, the first frequency is about 2.45 GHz (e.g., from about 2.402 GHz to about 2.480 GHz MHz), which is the frequency used by a Bluetooth low energy (BLE) device. The second frequency may be from about 1 MHz to about 1 GHz (e.g., 1 MHz, 2 MHz, 3 MHz, 4 MHz, 5 MHz, 6 MHz, 7 MHz, 8 MHz, 9 MHz, 10 MHz, 20 MHz, 30 MHz, 40 MHz, 50 MHz, 60 MHz, 70 MHz, 80 MHz, 90 MHz, 100 MHz, 200 MHz, 300 MHz, 400 MHz, 500 MHz, 600 MHz, 700 MHz, 800 MHz, 900 MHz, or 1 GHz). In some embodiments, the second frequency is about 915 MHz, about 433 MHz (e.g., about 433.050 MHz), or about 402 MHz (e.g., about 402-405 MHz). 433 MHz and 915 MHz correspond the industrial, scientific, and medical (ISM) RF frequencies, and 402 MHz corresponds to the Medical Implant Communications Systems (MICS) RF frequency. The topology of the systems described herein are shown in FIGS. 1 and 2.

The RF transceiver described herein can be housed in any module suitable for facilitating communication with a medical device (e.g., a medical device inserted into or implanted within the body or body cavity of a subject) and a peripheral device. In general, the transceiver is positioned external to the body and may be located, e.g., in close proximity, to the peripheral device. For example, the transceiver may be positioned less than 1 meter (e.g., less than 95 cm, 90 cm, 85 cm, 80 cm, 75 cm, 70 cm, 65 cm, 60 cm, 55 cm, 50 cm, 45 cm, 40 cm, 35 cm, 30 cm, 25 cm, 20 cm, 15 cm, 10 cm, 9 cm, 8 cm, 7 cm, 6 cm, 5 cm, 4 cm, 3 cm, 2 cm, 1 cm, or less) from the peripheral device. In some embodiments, the transceiver may be housed with, on, or adjacent to the peripheral device. The transceiver may be configured to fit within a pocket of a subject such that it is substantially stationary but is still located external to the body. In an embodiment in which the medical device is an intravaginal, intraurethral, or intrarectal device, the device may contain a cover or cap that protects the device when not in use. The cover or cap of the device may include the transceiver within the or on the surface of the cover or cap. Thus, when the user removes the cap or cover of the device, it can remain in the pocket of the user and positioned to communicate with the medical device and the peripheral device. The RF transceiver may be attached to, or integrated within, a peripheral device (e.g., a smartphone, computer, or tablet). For example, the relay device may be, e.g., be a micro-transceiver attached to the back of a user’s smartphone.

The transceiver may further include a power source (e.g., a battery). The power source can be used to operate one or more components of the transceiver, such as the microcontroller and the circuit board.

### Sensors

The RF transceivers described herein may be suitable for use in conjunction with any sensor technology, e.g., that is present on or in a medical device (e.g., a medical device inserted into or implanted within the body or body cavity of a subject). In some instances, the sensor is a microelectromechanical (MEM) sensor. The sensor may be an accelerometer, such as a multiple-axis accelerometer or a MEM accelerometer. In other instances, the sensor is a gyroscope, such as a multiple-axis gyroscope. The sensor may be a pressure sensor, muscle quality sensor, muscle strength sensor, biomolecule sensor (e.g., a hormone sensor and/or a toxin sensor), temperature sensor, moisture sensor, humidity sensor, electromyography (EMG) sensor, pH sensor, movement sensors, G-sensor, tilt sensor, rotation sensor, light detecting sensor (e.g., light detecting and ranging (LiDAR) sensor), electrical impedance myography (EIM) sensors. The medical device may contain one sensor or a plurality (e.g., 2, 3, 4, 5, 6, 7, 8, 9, 10, 15, 20, 25, 30, 35, 40, 45, 50, 100, or more) of sensors. The plurality of sensors may be of the same type or of differing types. For example, an intravaginal, intraurethral, or intrarectal device may include a plurality of positional sensors (e.g., MEM sensors, e.g., accelerometers) and at least one pressure sensor. An intraurethral device may further include a flow sensor. The device may further include a hall effect sensor.

The sensor is configured to transmit and/or receive a signal to and from the transceiver. Accordingly, the sensor may further include a transmitter, receiver, and/or a microcontroller in operative connection with the sensor. The sensor may be a transmitter and/or receiver.

In certain electronic systems, a microcontroller can connect to a sensor via a variety of wired connections, such as Serial-Peripheral Interface (SPI), Inter-Integrated Circuit (I2C), Universal Asynchronous Receiver/Transmitter (UART), or USB. A sensor (e.g., MEM accelerometer) may communicate with the MCU via 12C. The MCU and the ISM radio in the device may be on the same integrated chip. The radio can communicate with the relay device wirelessly, e.g., via the ISM band.

### Medical Devices

The RF transceivers described herein may be suitable for use in conjunction with any medical (e.g., biomedical) device that is fully or at least partially inserted into or implanted within the body of a subject. For example, the medical device may be any device that is located e.g., in an organ (e.g., heart, kidney, pancreas, brain, eye), muscle (e.g., leg, arm), or cavity (e.g., torso, stomach, bowel (e.g., colon, small intestine), mouth, anus, vagina, urethra, or rectum), and surrounded by some thickness of tissue. Exemplary implantable medical devices are implantable cardioverter defibrillators (ICDs), pacemakers, ostomy devices, retinal implants, smart contact lenses, glucose biosensors, cochlear implants, bladder implants or slings, implantable drug delivery systems, sleep apnea devices, wireless endoscopy capsules, vagal blocking devices, and electrical stimulation devices, e.g., for epilepsy, Parkinson’s disease and dystonia, pain relief and management, peripheral nerves, sacral nerves, phrenic nerves, lower esophagus. These and additional medical devices that may be used with the invention are described, e.g., Fitzpatrick, D. Implantable electronic medical devices. Elsevier, 2014, which is hereby incorporated by reference in its entirety.

Any device that includes a sensor for tracking biometric indicia may transmit the data from the sensor of the device to the peripheral device via the RF transceiver. For example, an ostomy device that senses a bowel pressure may include a pressure sensor that constantly monitors the bowel pressure in real time. When the bowel pressure goes above a predetermined threshold (e.g., 10 mmHg, 20 mmHg, 30 mmHg, 40 mmHg, 50 mmHg, 60 mmHg, 70 mmHg, 80 mmHg, 90 mmHg, 100 mmHg, or more), and the bowel needs to be evacuated, the peripheral device may provide an alert to the user (e.g., on the graphical user interface) to activate the bag of the ostomy device to fill, thereby reducing the bowel pressure. Other devices that may be used in conjunction with the systems and methods described herein include, e.g., intravaginal, intraurethral, and intrarectal devices, which are described in more detail below.

### Intravaginal Device

The medical device used in the systems and methods described herein may be an intravaginal device that contains one or more sensors (e.g., position or movement sensors). The intravaginal device may have an elongate shape (e.g., linear configuration) configured to fit within a female subject’s vagina. The device may have a shape that includes a substantially ring-shaped main body and a tether that extends from the main body. The intravaginal device may be used as part of a system for monitoring pelvic floor movements during, before, or after a daily activity or during a diagnostic procedure. The device can be inserted into the vagina of a female subject, such that the intravaginal device is positioned proximal to the cervix or vaginal cuff. The intravaginal device may contain one or more position or movement sensors (e.g., MEMS accelerometers) and/or other sensors. The positional and/or other sensors provide sensitive positional and/or other information that may be used to sensitively monitor pelvic floor movements and/or to assess the pelvic floor architecture or other health aspect of a subject.

In particular, the intravaginal device can be used during a daily activity or during a diagnostic procedure to detect patterns of angle change. For example, specific sensors in the intravaginal device can be monitored during the diagnostic testing to assess patterns and angle changes in the pelvic floor as a proxy for assessing the physiology of the pelvic floor muscles. The patterns and angle changes can be compared to those observed before a diagnosis or treatment in the tested subject or in subjects having a known pelvic floor disorder (e.g., urinary incontinence or pelvic organ prolapse) in order to accurately diagnose the pelvic floor disorder in the tested subject. The device may also be used to treat a female subject with a pelvic floor disorder. When a female patient performs a pelvic floor exercise with the device, the position of the pelvic floor musculature can be monitored during the exercise to ensure that she is performing the exercise correctly and maintaining activation of the pelvic floor muscles for a sufficient duration of time (e.g., a hold or lift).

Trends and patterns of angle changes of the sensors (e.g., MEMS accelerometers) observed in the intravaginal device during monitoring may be used to diagnose or predict a disease state based on the positions, movements, and relative orientation of the pelvic floor muscles (e.g., the various levator ani and anal sphincter muscle groups) and/or the pelvic floor organs or to assess the efficacy of a chosen therapy in the subject.

Exemplary intravaginal devices, systems, and methods for treating, training, visualizing, and diagnosing the health state of pelvic floor muscles of a subject have been extensively described in PCT Publication Nos. WO/2013/116310, WO/2015/103629, WO/2018/023037, WO/2019/084469, and WO/2019/084468, PCT Application No. PCT/US2019/027168, and U.S. Application No. 62/752,987, the disclosures of which are hereby incorporated by reference in their entirety.

The intravaginal device may have an elongate body configured to fit within the vagina. The intravaginal device may have a main body with an outer edge configured to contact all or a portion of the vaginal wall surrounding the cervix or vaginal cuff and has an internal diameter sized to approximately circumferentially surround a cervix or a vaginal cuff. The internal and external diameter of the intravaginal device may be approximately equivalent, with the difference in their length being attributable to the thickness of the material used to fabricate the intravaginal device. The internal and/or external diameter may be about 20 mm to about 80 mm (e.g., about 20, 25, 30, 35, 40, 45, 50, 55, 60, 65, 70, 75, or 80 mm) in length. In some instances, the internal diameter of the intravaginal device may be smaller than the external diameter. In some instances, the intravaginal device can be fabricated with a tether (e.g., a flexible cord or ribbon) that can be optionally attached, e.g., by a removable or permanent connection, to the main body of the intravaginal device, The tether can have a length of up to about 14 cm (e.g., 1, 2, 3, 4, 5, 6, 7, 8, 9, 10, 11, 12, 13, or 14 cm) and a width of about 1 to about 10 mm (e.g., 1, 2, 3, 4, 5, 6, 7, 8, 9, or 10 mm). Different form factors of the device include a ring (round or oval), a ring with a tether, and an incomplete ring (e.g., a horseshoe configuration). In some embodiments, the device may not have a ring, and the device is substantially linear or elongate.

The outer edge of the main body of the intravaginal device may be configured to apply pressure, tension, adhesion, and/or suction to the vaginal wall to hold the position of the intravaginal device at a location proximal to the cervix or vaginal cuff of the subject. The pressure, tension, adhesion, and/or suction applied to the vaginal wall by the outer edge of the intravaginal device is of a sufficient strength to limit slippage, repositioning, or displacement of the intravaginal device from the vaginal canal of the subject.

Additionally, the main body of the intravaginal device may include one or more (e.g., 1, 2, 3, 4, 5, 6, 7, 8, 9, 10, or more) features for the purpose of stabilizing, orienting, and/or positioning the device within the body of the subject. The feature may be selected from the group consisting of a coating, a protrusion, and a texture. In some instances, the feature is a coating (e.g., a surface coating) containing one or more one (e.g., 1, 2, 3, 4, 5, 6, 7, 8, 9, 10, or more) biomaterials. The retention features may be applied as in the devices shown or they can be applied as features to other devices described herein, The retention features may be useful for a device of the invention that is designed to remain inside a woman’s vagina for an extended period of time (e.g., at least 10 minutes, 20 minutes, 30 minutes, 40 minutes, 50 minutes, 1 hour, 2 hours, 3 hours, 4 hours, 5 hours, 6 hours, 12 hours, 24 hours, 2 days, 3 days, 4 days, 5 days, 6 days, 1 week, 2 weeks, 3 weeks, 4 weeks, 1 month, 2 months, 3 months, 4 months, 5 months, 6 months, 7 months, 8 months, 9 months, 10 months, 11 months, 12 months).

The intravaginal device includes one or more (e.g., 1, 2, 3, 4, 5, 6, 7, 8, 9, 10, 20, or more) sensors within the main body (e.g., the substantially ring-shaped form) and/or the tether that are configured to detect a muscle movement, e.g., a PFL and/or a PFR. In some instances, the sensor may be configured to detect a muscle movement, e.g., a PFL and/or a PFR, in substantially real-time. In some instances, the sensors (e.g., 1, 2, 3, 4, 5, 6, 7, 8, 9, 10, 20, or more sensors) may be selected from the group consisting of a movement sensor, an orientation sensor, an accelerometer, a gyroscope, a micro-electro-mechanical systems (MEMS) sensor (e.g., MEMS accelerometer), a G-sensor, a tilt sensor, a rotation sensor, a pressure sensor, a light detecting sensor, such as a LiDAR sensor, an EIM sensor, and combinations thereof. The device may also include a light generating component for use with the light detecting sensor, such as a LiDAR sensor. The device may also include an electrode for use with the EIM sensor. Additionally, the intravaginal device may include one or more sensors (e.g., 1, 2, 3, 4, 5, 6, 7, 8, 9, 10, 20, or more sensors) configured to detect, e.g., a level of or change in the level of muscle strength, muscle quality, a biomolecule (e.g., a hormone and/or a toxin), pH, temperature, and/or humidity.

In some instances, the sensors may be positioned in an arrangement similar to or in an arrangement different from those described in, e.g., International Publication Nos. WO2015/103629, WO2016/067023, and WO2016/042310; U.S. Publication Nos. US20150032030, US20140066813, US20150151122, US20150133832, US20160008664, and US20150196802; and U.S. Pat. Nos. US8983627, US7955241, US7645220, US7628744, US7957794, US6264582, and US6816744, each of which is incorporated by reference herein. For example, two or more sensors, as described herein, may be placed around the longitudinal axis of the intravaginal device, e.g., in a circle or a spiral around the central-axis of the main body and/or tether of the intravaginal device, approximately at ± 1°, 2°, 3°, 4°, 5°, 6°, 7°, 8°, 9°, 10°, 20°, 30°, 40°, 50°, 60°, 70°, 80°, 90°, 100°, 110°, 120°, 130°, 140°, 150°, 160°, 170°, 180°, 190°, 200°, 210°, 220°, 230°, 240°, 250°, 260°, or 270° relative to each other. Alternatively, or additionally, two or more sensors, as described herein, may be placed approximately 0.001 mm, 0.01 mm, 0.1 mm, 0.5 mm, 1 mm, 2 mm, 3 mm, 4 mm, 5 mm, 6 mm, 7 mm, 8 mm, 9 mm, 10 mm, 20 mm, 30 mm, 40 mm, 50 mm, 60 mm, 70 mm, 80 mm, 90 mm, 100 mm, 125 mm, 150 mm, 175 mm, 200 mm, 225 mm, 250 mm, 275 mm, 300 mm, 325 mm, 350 mm, or more apart, e.g., along the circumference of the main body and/or along the length of the device or the length of the tether of the intravaginal device. In some instances, the two or more sensors, as described herein, may be placed along the central-axis of the main body and/or tether of the intravaginal device. In some instances, the two or more sensors, as described herein, may be placed such that they are not on the central-axis, e.g., such that they are offset from the central axis of the main body and/or tether of the intravaginal device. In particular instances, such as when sensors are positioned within the tether, the main body may not contain a sensor. In other instances, when sensors are positioned within the tether the main body may also contain one or more (e.g., 1, 2, 3, 4, 5, 6, 7, 8, 9, 10, 20, or more) sensors. In some instances, the sensor is an accelerometer, such as a multiple-axis accelerometer. In other instances, the sensor is a gyroscope, such as a multiple-axis gyroscope. In yet other instances, the sensor is a MEMS sensor. Additionally, the intravaginal device may further include one or more (e.g., 1, 2, 3, 4, 5, 6, 7, 8, 9, 10, 20, or more) additional sensors within the main body and/ or the tether selected from the group consisting of a pressure sensor, a muscle quality sensor, a muscle strength sensor, a biomolecule sensor (e.g., a hormone sensor and/or a toxin sensor), a temperature sensor, a moisture sensor, a humidity sensor, an electromyography (EMG) sensor, and a pH sensor. A sensor(s) can be positioned on the surface of the intravaginal device (e.g., on the surface of the main body and/or tether), such that all or a portion of the sensor(s), makes direct contact with the tissues of the vaginal walls and/or cervix or vaginal cuff of a subject. In some instances, the sensor(s) can be positioned about 0.001 mm, 0.01 mm, 0.1 mm, 0.2 mm, 0.3 mm, 0.4 mm, 0.5 mm, 0.6 mm, 0.7 mm, 0.8 mm, 0.9 mm, 1 mm, 1.5 mm, 2 mm, 2.5 mm, 3 mm, 3.5 mm, 4 mm, 4.5 mm, 5 mm, or more below the exterior surface (e.g., the surface that makes direct contact with the tissues of the vaginal walls and/or cervix or vaginal cuff of a subject) of the intravaginal device (e.g., the main body and/or tether of the intravaginal device). In some instances, the sensor can be positioned such that about 0.001 mm, 0.01 mm, 0.1 mm, 0.2 mm, 0.3 mm, 0.4 mm, 0.5 mm, 0.6 mm, 0.7 mm, 0.8 mm, 0.9 mm, 1 mm, 1.5 mm, 2 mm, 2.5 mm, 3 mm, 3.5 mm, 4 mm, 4.5 mm, 5 mm, or more of the sensor protrudes from the exterior surface of the intravaginal device (e.g., the main body and/or tether of the intravaginal device). Alternatively, the sensors can be positioned within the intravaginal device (e.g., within the main body and/or tether), such that the sensor does not directly contact the vaginal walls and/or cervix or vaginal cuff of a subject but is positioned to detect motion as the user conducts a PFL or PFR. The sensor(s) may be evenly or unevenly positioned at intervals on or within the intravaginal device. The sensors within the intravaginal device (e.g., within the main body and/or tether) may be positioned such that when the intravaginal device is inserted into a user the sensors face the ventral direction (e.g., anterior direction).

The tether can be up to about 20 cm (e.g., 1, 2, 3, 4, 5, 6, 7, 8, 9, 10, 11, 12, 13, 14, 15, 16, 17, 18, 19, or 20 cm) in length and may be divided along its length into segments contain sensors. Sensors can be positioned along the length of the tether at even or uneven intervals, e.g., at an interval of about 1 to about 140 mm (e.g., 1, 2, 3, 4, 5, 6, 7, 8, 9, 10, 20, 30, 40, 50, 60, 70, 80, 90, 100, 110, 120, 130, or 140 mm). The location of a sensor within the tether may be identified on the outside of the device by the presence of indicia (e.g., a protrusion, symbol, writing, and/or etching) on the surface of the tether.

The intravaginal device (e.g., main body (e.g., the substantially ring-shaped form) and/or tether) further includes a microcontroller, e.g., within the substantially ring-shaped form that is configured for receiving data from the sensor(s). The microcontroller may also be configured, or can include a separate component, for non-transiently storing data from the sensor(s). The microcontroller may be connected to the sensor(s), e.g., by a wire and/or a circuit board. The wire and circuit board may be flexible or rigid.

The intravaginal device can also include a transmitter and receiver within main body (e.g., the substantially ring-shaped form) and/or tether form for communicating wirelessly or via a detachable cable with a peripheral device (e.g., a handheld or portable device or a computer, such as a smartphone, tablet, or laptop), e.g., via the relay device. Alternatively, the transmitter and receiver may be located in an external housing and connected to the intravaginal device wirelessly or by a detachable cable. The transmitter and receiver can be connected directly or indirectly to the microcontroller, sensor(s), and/or circuit board. The transmitter and receiver are configured for use with a RF transceiver as described herein. The transmitter and receiver may communicate with the transceiver e.g.., using BLE, ISM, MICS, Wi-Fi, or RF. Information collected by the sensor(s) may be communicated (e.g., downloaded, transferred) to the peripheral device wirelessly by the transmitter and receiver, e.g., via the RF transceiver relay device, and/or by using the detachable cable.

In certain embodiments, intravaginal device contains 8 or fewer (e.g., 4 or 5) sensors in the tether and 5 or fewer sensors in main body. One sensor may be shared by both the tether and main body. The angle between the plane connecting the anterior and posterior aspects of the main body 110 and tether 10 may vary from 0° - 180° (e.g., 10°, 20°, 30°, 40°, 50°, 60°, 70°, 80°, 90°, 100°, 110°, 120°, 130°, 140°, 150°, 160°, 170°, 180°). The circumference of the main body may be from about 10 cm to about 50 cm (e.g., 15 cm, 20 cm, 25 cm, 30 cm, 35 cm, 40 cm, 45 cm, or 50 cm) or may be 27.6 cm. The length of tether 10 may be from about 1 cm to about 50 cm (e.g., 2 cm, 3 cm, 4 cm, 5 cm, 10 cm, 15 cm, 20 cm, 25 cm, 30 cm, 35 cm, 40 cm, 45 cm) or may be 25.5 cm long. The sensors 200 may be spaced about 0.5 cm to about 5 cm (e.g., 1 cm, 1.5 cm, 2 cm, 2.5 cm, 3 cm, 3.5 cm, 4 cm, or 4.5 cm) or may be spaced about 1.6 cm apart. At least one sensor may be placed on the tether 10 cm or less (e.g., 9 cm, 8 cm, 7 cm, 6 cm, 5 cm, 4 cm, 3 cm, 2 cm, or 1 cm) from the main body.

The intravaginal device may further include a power source (e.g., a battery). The power source can be used to operate one or more components of the device, such as the sensor(s), transmitter, receiver, and the circuit board.

### Intraurethral Device

The medical device used in the systems and methods described herein may be an intraurethral device that includes at least one sensor. The intraurethral device may be a catheter, such as a urodynamic catheter. The device may have an elongated main body and one or more position or movement sensors (e.g., MEMS accelerometers) positioned along a length thereof. The catheter can be used alone or as part of a system for monitoring pelvic floor movement. The catheter is configured for insertion into the urethra of a subject (e.g., male or female subject), such that the one or more sensors (e.g., positional sensors) provide data, such as positional data that provide readout of the spatial orientation of the subject’s urethra. The position of the urethra provides a readout that acts as a proxy for the pelvic floor and the spatial arrangement of the pelvic floor organs, including the urethra. For a male subject, the catheter orientation can provide information on the position of the prostate. The catheter may also include one or more additional sensors, such as movement, pressure, and/or flow sensors. The catheter may be structurally configured of any suitable geometry to fit within a subject’s urethra. The catheter may have multiple, e.g., 2 lumens. One lumen may be for filling the bladder and one lumen may be for measuring pressure.

Exemplary catheters that can be used in conjunction with the devices, systems, and methods described herein are described, for example, in PCT Publication Nos. WO/2011/050252 WO/2013/082006, the disclosures of which are hereby incorporated by reference in their entirety. Exemplary urodynamic catheters that can be used, and/or modified with an additional sensor to are described, for example, in U.S. Pat. Nos. US6447462 and US5984879, and in U.S. Publication Nos. US20060122488, US20030097039, US20060276712, US20060281992, and US20170258345, the disclosures of which are hereby incorporated by reference in their entirety.

The intraurethral device may include a balloon. For example, the device may include at least one thin-walled, circumferentially-extending balloon proximate the distal, or subject end thereof which communicates pressure external to the balloon proximally to a transducer external to the subject’s body through a small-volume, closed air column. The catheter can be inserted with the at least one balloon in a collapsed state and expanded after entry of the catheter into the subject’s bladder during a multi-channel cystometry procedure.

The intraurethral device may include one or more (e.g., 1, 2, 3, 4, 5, 6, 7, 8, 9, 10, 20, or more) sensors, such as position or movement sensors. The position or movement sensors may be configured to detect a muscle movement, e.g., a PFL and/or a PFR. In some instances, the sensors may be configured to detect a muscle movement, e.g., a PFL and/or a PFR, in substantially real-time. In some instances, the one or more sensors may be selected from the group consisting of a movement sensor, an orientation sensor, an accelerometer, a gyroscope, a micro-electro-mechanical systems (MEMS) sensor (e.g., MEMS accelerometer), a G-sensor, a tilt sensor, a rotation sensor, a pressure sensor, a temperature sensor, a moisture sensor, an electromyography (EMG) sensor, a light detecting sensor, such as a LiDAR sensor, an EIM sensor, and combinations thereof.

Two or more sensors, as described herein, may be placed around the longitudinal axis of the catheter, e.g., in a circle or a spiral around the central-axis of the main body and/or tether of the catheter, approximately at ± 1°, 2°, 3°, 4°, 5°, 6°, 7°, 8°, 9°, 10°, 20°, 30°, 40°, 50°, 60°, 70°, 80°, 90°, 100°, 110°, 120°, 130°, 140°, 150°, 160°, 170°, 180°, 190°, 200°, 210°, 220°, 230°, 240°, 250°, 260°, or 270° relative to each other. Alternatively, or additionally, two or more sensors, as described herein, may be placed approximately 0.001 mm, 0.01 mm, 0.1 mm, 0.5 mm, 1 mm, 2 mm, 3 mm, 4 mm, 5 mm, 6 mm, 7 mm, 8 mm, 9 mm, 10 mm, 20 mm, 30 mm, 40 mm, 50 mm, 60 mm, 70 mm, 80 mm, 90 mm, 100 mm, 125 mm, 150 mm, 175 mm, 200 mm, 225 mm, 250 mm, 275 mm, 300 mm, 325 mm, 350 mm, or more apart, e.g., along the of the catheter. In some instances, two or more sensors, as described herein, may be placed along the central-axis of catheter. In some instances, two or more sensors, as described herein, may be placed such that they are not on the central-axis, e.g., such that they are offset from the central axis of the catheter. A sensor(s) can be positioned on the surface of the catheter, such that all or a portion of the sensor(s), makes direct contact with the tissues of the urethra. In some instances, the sensor(s) can be positioned about 0.001 mm, 0.01 mm, 0.1 mm, 0.2 mm, 0.3 mm, 0.4 mm, 0.5 mm, 0.6 mm, 0.7 mm, 0.8 mm, 0.9 mm, 1 mm, 1.5 mm, 2 mm, 2.5 mm, 3 mm, 3.5 mm, 4 mm, 4.5 mm, 5 mm, or more below the exterior surface (e.g., the surface that makes direct contact with the tissues of the urethra) of the catheter. In some instances, the sensor can be positioned such that about 0.001 mm, 0.01 mm, 0.1 mm, 0.2 mm, 0.3 mm, 0.4 mm, 0.5 mm, 0.6 mm, 0.7 mm, 0.8 mm, 0.9 mm, 1 mm, 1.5 mm, 2 mm, 2.5 mm, 3 mm, 3.5 mm, 4 mm, 4.5 mm, 5 mm, or more of the sensor protrudes from the exterior surface of the catheter. Alternatively, the sensors can be positioned within the catheter, such that the sensor does not directly contact the urethra, but are positioned to detect motion, e.g., during a pelvic floor movement. The sensor(s) may be evenly or unevenly positioned at intervals on or within the catheter. The sensors within the catheter may be positioned such that when the catheter is inserted into a subject the sensors face the ventral direction (e.g., anterior direction).

The catheter can also include a transmitter and/or receiver for communicating wirelessly or via a detachable cable with an electronic device (e.g., a peripheral device, such as a handheld or portable device or a computer, such as a smartphone, tablet, or laptop). Alternatively, the transmitter and receiver may be located in an external housing and connected to the catheter wirelessly or by a detachable cable. The transmitter and receiver can be connected directly or indirectly to the microcontroller, sensor(s), and/or circuit board. The transmitter and receiver are configured for use with a RF transceiver as described herein. The transmitter and receiver may communicate with the transceiver e.g.., using BLE, ISM, MICS, Wi-Fi, or RF. Information collected by the sensor(s) may be communicated (e.g., downloaded, transferred) to the peripheral device wirelessly by the transmitter and/or receiver and/or by using the detachable cable. The peripheral device can include a user interface. The user interface can be programmed to display data and/or to provide instructions for use of the intraurethral device (e.g., catheter, e.g., urodynamic catheter). The intraurethral device may further include a power source (e.g., a battery). The power source can be used to operate one or more components of the device, such as the sensor(s), transmitter, receiver, and the circuit board.

### Intrarectal Device

The medical device may be an intrarectal device that includes at least one sensor. The intrarectal device has an elongated main body and can be used as part of a system for monitoring pelvic floor movements during. Exemplary intrarectal devices are described, for example, in U.S. Publication Nos. US20170281072 and US20170303843, the disclosures of which are hereby incorporated by reference in their entirety.

The device can be inserted into the rectum of a subject (e.g., male or female subject), such that the one or more sensors (e.g., position or movement sensor) provide data, e.g., in real-time. For example, a position or movement sensor may provide a positional readout of the spatial orientation of the subject’s rectum. The position of the rectum provides a readout that acts as a proxy for the position of the pelvic floor and the spatial arrangement of the pelvic floor organs, including the urethra and prostate. The intrarectal device may include a plurality of positional sensors (e.g., accelerometers) positioned along a length of the device. The intrarectal device may also include one or more additional sensors, such as movement and/or pressure sensors. The intrarectal device may be structurally configured of any suitable geometry to fit within a subject’s rectum.

The intrarectal device can be used to detect patterns of angle change. For example, specific sensors in the intrarectal device can be monitored during urodynamic testing or a daily activity to assess patterns and angle changes in the pelvic floor as a proxy for assessing the physiology of the pelvic floor muscles. The patterns and angle changes can be compared to those observed before a diagnosis or treatment in the tested subject or in subjects having a known pelvic floor disorder (e.g., urinary incontinence) in order to accurately diagnosis the pelvic floor disorder in the tested subject.

Trends and patterns of angle changes of the sensors (e.g., MEMS accelerometers) observed in the intrarectal device, e.g., during urodynamic testing may be used to diagnose or predict a disease state based on the positions, movements, and relative orientation of the pelvic floor muscles (e.g., the various levator ani and anal sphincter muscle groups) and/or the pelvic floor organs or to assess the efficacy of a chosen therapy in the subject.

### Peripheral Device

The systems and methods described herein may include a peripheral device. The peripheral device may be any suitable electronic device, such as a computer, smartphone, tablet, or smart watch. The peripheral device may be programmed with a software or mobile application to facilitate use in conjunction with the devices and systems described herein. The peripheral device may be configured with a processing unit that can transform or utilize sensor data received from the medical device (e.g., a medical device inserted into or implanted within the body or body cavity of a subject, e.g., an intraurethral, intrarectal, or intravaginal device). For example, the sensor data may be received when a subject performs a pelvic floor movement, such as during a daily activity (e.g., activity that alters (e.g., increases and/or decreases) the overall health of her urogenital system and/or pelvic floor), to provide feedback to the subject regarding whether the detected activity affects her health status or is indicative of treatment of, or a need for treatment for, a pelvic floor disorder, such as urinary and/or fecal incontinence. For example, the peripheral device can process the sensor data to produce a baseline that can be used for comparison to sensor data obtained at a future time to provide feedback to the subject (e.g., an alert) regarding whether activities she performs are beneficial or detrimental to her health status or whether the pelvic floor movements are indicative of treatment of, or a need for treatment for a pelvic floor disorder. In addition, or alternatively, the peripheral device can process the sensor data and compare the result to a previously established or predetermined baseline and based on the comparison can provide feedback to the subject (e.g., an alert) regarding whether activities performed are beneficial or detrimental to her health status or whether the pelvic floor movements are indicative of treatment of, or a need for treatment for a pelvic floor disorder. Additionally, the peripheral device can include a user interface. The user interface can be programmed to display data and/or to provide instructions for use of the medical device.

The peripheral device is configured to transmit and/or receive a signal to and from the transceiver. Accordingly, the peripheral device may further include a transmitter, receiver, and/or a microcontroller in operative connection with the peripheral device. The peripheral device may be a transmitter and/or receiver.

The peripheral device may be equipped with a Wi-Fi or internet connection. For example, the peripheral device may be able to transmit the data from the sensors to a cloud-based, web-based, server, or other information storage regime.

### Systems and Kits

The devices and components described herein may be present as part of a kit or system. For example, the systems and kits described herein may include one or more of the radio frequency transceiver, a medical device (e.g., a medical device inserted into or implanted within the body or body cavity of a subject, e.g., intravaginal, intraurethral, or intrarectal device), a peripheral device (e.g., smartphone), one or more sensors, microcontrollers, transmitters, receivers, and the like (see, e.g., FIG. 2). If packaged in a kit, the kit may further include instructions for use thereof. If the medical device is, e.g., an intravaginal device, the system or kit may further include a cap or cover for the device. The cap or cover may optionally include the RF transceiver described herein. The peripheral device (e.g., smartphone) may further include a mobile application or web-based application.

### Operational Use

Transmission of data from a sensor implanted within the human to a peripheral device is a very desirable approach from a usability perspective. However, RF attenuation within the body and a mismatch in technologies may prohibit direct connectivity that would otherwise be desired. Instead, a hybrid approach utilizing a seamless bridging device may be deployed to allow for heterogenous usage of RF, with the embedded sensor operating on an RF band frequency that is significantly more transmissive through the human body, while the peripheral device operates on a convenient RF band used for commercial accessories.

Medical sensors in a device inserted into or implanted within the body typically need to communicate wirelessly with external devices in order to transmit data in real time. A common interface device available today to patients is the Smartphone, with Bluetooth Low Energy (BLE) being the predominant wireless interface (see, e.g., FIG. 1).

### Link-Budget Analysis

A wireless link is composed of at least one transmitter and one receiver. For two-way communication, each side requires one transmitter and one receiver. The signal power received by each receiver is calculated using the following equation:

$$\begin{matrix}\text{Received Power} \left(\text{dBm}\right) = \text{Transmitted Power} \left(\text{dBm}\right) + \\ \text{Gains} \left(\text{dB}\right) - \text{Losses} \left(\text{dB}\right)\end{matrix}$$

Effective wireless communication requires that the received signal power at each end of the wireless link be sufficiently larger than the noise input power. The minimum received signal strength that results in acceptable bit error rates is referred to as the sensitivity of a receiver. The sensitivity of a receiver is a function of several factors. These include, the noise performance of the system electronics, the efficiency of the receiving antenna, and the modulation used to encode data onto an RF carrier. Choosing the right modulation scheme requires tradeoffs between complexity, data rates, channel bandwidth, and bit error rates for a given signal-to-noise ratio.

The primary losses in a typical wireless system are channel losses. Free-space loss is caused primarily by the spreading of energy in all directions such that only a small amount of the energy radiated by the transmitter antenna reaches the receiving antenna. For biomedical systems in which one or both antennas are surrounded by human tissue, a second major source of loss is caused by tissue absorption. The conductivity and permittivity characteristics of human tissue cause it to absorb RF energy and convert it to heat, resulting in significantly higher amounts of loss than free space.

### Connection - BLE and ISM

In order to ensure a transparent bridging of the ISM and BLE transport segments, it may be necessary to synchronize the establishment and teardown of the two links. Since BLE is established from the peripheral device acting as a BLE Central, the ISM link establishment may be governed by the peripheral device (e.g., smartphone) BLE connection. Upon establishment of a BLE connection, the ISM link is established. Upon teardown of an existing BLE connection, the ISM link may also need to be torn down.

The ISM link may not need to govern the BLE connection itself. In case of loss of ISM data, a higher-level protocol can be used to determine what should take place on the BLE link. In addition to the formation of the actual connection, the underlying nature of the two transports may need to be aligned to allow for the smooth exchange of data between them. The approach described herein logically arranges each transport to appear to be a full-duplex simple serial pipe. This would allow bounded data packets to be received on one transport and simple sent on the other unmodified.

### Data Exchange - Packet Based, Encoding

Once a BLE connection and ISM link are established, data can flow between the two endpoints, the sensor, and the peripheral device (e.g., smartphone), with the RF transceiver bridging the two transports. ISM packets may be transparently reconstructed and sent on BLE connection. Similarly, packets received at the RF transceiver relay device that are destined for the sensor may be reconstructed and sent on ISM link. Both transports may utilize a simple Consistent Overhead Byte Stuffing (COBS) encoding to encapsulate the underlying data packets. COBS is an algorithm for encoding data bytes that results in efficient, reliable, and unambiguous packet framing regardless of packet content, thus making it easy for receiving applications to recover from malformed packets. It employs a particular byte value (e.g., zero) to serve as a packet delimiter, which is a special value that indicates the boundary between packets. When zero is used as a delimiter, the algorithm replaces each zero data byte with a non-zero value so that no zero data bytes will appear in the packet and thus be misinterpreted as packet boundaries.

### Energy Management

One additional constraint for the overall system is the management of energy, such as the battery within the sensor or the device. This is critical as the sensor may be implanted by a physician and may not be readily removable. As such, it is necessary to ensure the operation of the sensor for the expected treatment duration. To achieve this, the sensor may maintain itself in a resting mode, e.g., deep sleep mode, to conserve energy when not in use. The sensor may wake up periodically to check for activation briefly and return to sleep if no activation pulse is observed. This may take place over the ISM link where the sensor may monitor for the existence of a transmission with a unique signature specific to the sensor.

In order to affect wakeup of the sensor, the matching relay will emit an activation pulse with a duration, e.g., of 10%, longer than the sensor wakeup period. This may ensure that the next time the accompanying sensor checks for activation, it will find it. Once activated, the sensor collects and sends data, pausing in-between transmissions for an acknowledgement from the accompanying relay. The lack of acknowledgement forces the sensor to abandon further transmissions and to return back to a resting mode, e.g., deep sleep mode, awaiting the next activation.

The medical device (e.g., a medical device inserted into or implanted within the body or body cavity of a subject, such as, e.g., an intravaginal, intraurethral, or intrarectal device) may include a hall effect sensor. A hall effect sensor may be used to detect when the device is activated (e.g., removed from its case) in order to trigger the microcontroller and/or the radios within the transceiver and/or the medical device. The sensor may also be used to detect when the device is deactivated, e.g., the case is put back on. The hall effect sensor has omnipolar detection of magnetic flux. Its output will be at its VCC (voltage at the common collector) level until it experiences magnetic flux greater than 4.8 mT in magnitude, after which it will switch its output to GND (ground). Its output will return to VCC once the magnetic field is reduced or removed. The microcontroller may check the level of the hall effect sensor output once a second to determine its state. The state of the hall effect sensor may be used to determine whether the device should be in active operation or standby. For example, if a magnetic field is no longer detected, the device may be outside of the case so the device may enter its active mode. Conversely, the presence of a magnetic field may indicate that the device is within the case and the system should enter or remain in standby mode.

### Pelvic Floor Disorders

The devices, systems, and kits of the invention may be used to monitor and diagnose pelvic floor disorders. Pelvic floor disorders include urinary tract disorders, which are disorders that impart difficulties in bladder storage, and urinary incontinence, which includes an inability of the body to control the discharge of urine. Types and prevalence of incontinence among ambulatory adult women include stress urinary incontinence (SUI), detrusor instability (urge incontinence), mixed incontinence (stress and urge), and other incontinence (overflow, neurogenic). The prevalence of detrusor muscle instability and of mixed incontinence has been observed to increase with age of the subject sample. Male subjects may experience similar incontinence problems, which are often associated with an enlarged prostate gland. Males also have urine retention issues due to the prostate.

SUI may be characterized by involuntary loss of urine occurring when, in the absence of a detrusor contraction, intravesical pressure exceeds maximum urethral pressure. Stress urinary incontinence may include accidental loss of urine resulting from laughing, sneezing, coughing, or standing up, as any such exertion causes increased abdominal pressure, as transmitted to the bladder and the urine contained therein, to exceed the resistance to flow generated by the urethra, and principally the urethral sphincter. SI may be further categorized as hypermobility of the bladder neck and intrinsic sphincteric deficiency (ISD).

Hypermobility of the bladder neck may result from descent of the pelvic floor and may be attributed to weakened pelvic floor muscles and connective tissue. This may be observed in combination with nerve damage to the external genitalia resulting from childbirth but may also occur in younger women who have not given birth. In a normal position, the bladder is supported by the pelvic muscles, which prevent increases in abdominal pressure from exceeding urethral pressure. When the pelvic muscles are weakened or damaged, the bladder neck is abnormally displaced during abdominal stress and the urethral sphincter closure pressure becomes inadequate to maintain continence. Loss of urine due to hypermobility-related SI typically occurs in a periodic manner and the volume of urine may be proportional to the severity of the condition.

ISD is a severe form of stress incontinence which may occur due to an intrinsic deficiency of the urethral closure mechanism or due to a dysfunctional urethra where the bladder neck is open at rest. Severe ISD results in continuous leakage of urine or leakage responsive to only minimal subject exertion. In ISD, the bladder neck may be fixed, or hypermobile. ISD occurs in a significant number of instances due to urethral scarring from past incontinence surgeries but may result from other causes. Only a small number of subjects exhibit stress incontinence attributable to ISD.

### EXAMPLES

The following examples are put forth to provide those of ordinary skill in the art with a description of how the components and methods described herein may be used, made, and evaluated, and are intended to be purely exemplary of the invention and are not intended to limit the scope of what the inventors regard as their invention.

### Example 1. Simulation of Implanted Sensor in Phantom Solution

A phantom simulation in a water-based solution was used to simulate communication between a sensor and a peripheral device in human tissue in performing bench testing. The phantom solution was built using the following process:
A. Heat up approximately 3 gallons (+/- 0.1 gal.) of water to 104° F. (+/- 1° F.)
B. Fill a 5-gallon bucket with the warm water.
C. Add 22 lbs. (+/- 1 lb.) of sugar (sucrose) to the bucket while stirring.
D. Add 10.6 oz (+/- 0.1 oz) of salt to the bucket while stirring.
E. Use the temperature stabilization device to heat up the water to 98° F. (+/- 2° F.)

Two prototypes were designed and prototyped to compare the RF performance of a BLE radio and a 915 MHz ISM radio. The radios were configured to transmit continuous wave signals at similar output power values without any modulation. A receiver was located approximately 30 cm from the prototype transmitter, and the received power was as follows:

Received signal strength for BLE radio in free space: -53 dBm.

Received signal strength for ISM radio in free space: -50 dBm.

Each prototype transmitter was then submerged in the phantom solution and the received power was measured:

Received signal strength for BLE radio in phantom: less than -101 dBm.

Received signal strength for ISM radio in phantom: -85 dBm.

The sensitivity of the BLE receiver was approximately -101 dBm. The received signal strength was below the sensitivity limit, so the attenuation due to the phantom solution was at least 48 dB (difference between -53 dBm and -101 dBm). For the ISM radio, the attenuation was 35 dB (difference between -85 dBm and -50 dBm). The phantom solution experiment showed that the 915 MHz radio signals experienced at least 18 dB less attenuation in phantom solution than the BLE radio signals.

### Example 2. Sensor Device Testing in a Human

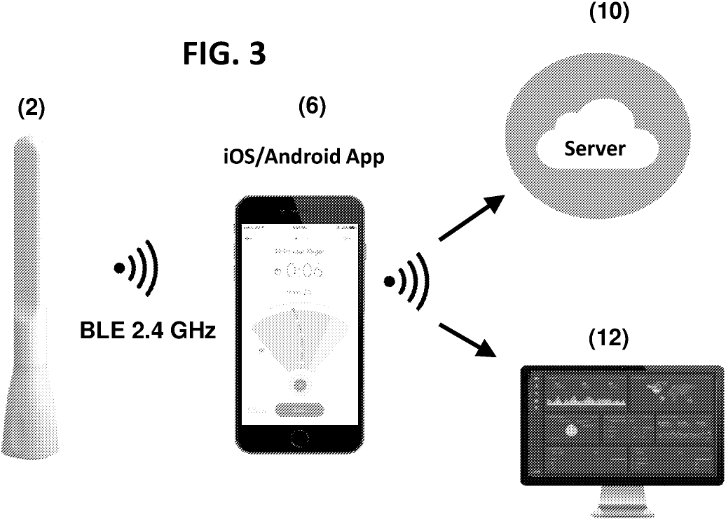

A system that includes a smartphone (6) and an intravaginal device (2) with 6 accelerometers located along a length of the device was tested in four human subjects with four separate prototypes. This system did not include a relay device (FIG. 3). All four prototypes functioned properly in free space. However, when the devices were inserted into each subject’s vagina, the wireless connection was dropped. This confirmed the phantom testing indicating that the attenuation of BLE signals is attenuated to a level below the sensitivity of a typical BLE receiver when placed inside the human body.

An updated system that includes a relay device (8) (FIGS. 1 and 2) was subsequently tested in humans. The relay device was comprised in the case of the intravaginal device and includes two wireless radios: a 915 MHz band ISM radio and a 2.4 GHz BLE radio. The BLE communicates to a mobile phone via BLE. The system was tested with two modulation schemes that were as follows:

WB-DSSS: 60 kbps, 195 kHz deviation, 2-GFSK, 4x spreading.

GFSK: 50 kbps, 25 kHz deviation, 2-GFSK, no spreading.

The ISM radio allows various RF settings to be adjusted to enable tradeoffs based on the specific requirements of an application. These include transmitter output power, channel frequencies, modulation schemes, data rates, and filter bandwidths. The maximum output power achievable by the ISM radio used is +15 dBm. This output power level, however, consumes considerable amounts of current. FCC rules described in Part 15 of Title 47 set a variety of limits to various metrics including radiated power and occupied bandwidth. Specifically, Part 15.247 defines rules for devices that employ frequency hopping and digital modulation, whereas Part 15.249 does not enforce restrictions on modulation schemes but limits the maximum permitted field strength to a lower limit. To meet the rules of FCC guidelines for Part 15.249, the maximum permitted field strength is 50 mV/m, which is approximately equivalent to -1 dBm of conducted power at the antenna port.

Initial prototypes of the system used an output power of +5 dBm. Since this exceeds the maximum output power for Part 15.249 of FCC guidelines, operating at this power level requires the use of frequency hopping, digital modulation, and a 6 dB bandwidth exceeding 500 kHz. To achieve these requirements, a Wide-Band Direct Sequence Spread Spectrum (WB-DSSS) scheme was implemented in the ISM radios along with a Gausian Frequency Shift Keying (GFSK) modulator. Using WB-DSSS resulted in a wide enough band that the FCC requirements could be met. However, the wider bandwidth resulted in a higher noise floor, resulting in suboptimal error rates during human testing.

A total of 15 human subjects were given a device using the WB-DSSS modulation and asked to use it at home. Depending on the subject and their environment, an average of 14.65% of sessions resulted in a connection failure. A connection failure was the result of 1 second worth of data packets that had at least one data bit error. The results comparing the percentage of connection failures between a system without the relay device and a system with the relay device is shown in Table 1 below. While this modulation scheme resulted in some errors, the overall performance was substantially better than the system in FIG. 3 that attempts to communicate directly with the peripheral device using a 2.4 GHz BLE radio.

A second modulation scheme was also tested that limited the output power at the antenna to -1 dBm and did not employ frequency hopping. As shown in Table 1, the GFSK modulation resulted in substantially fewer connection errors than the WB-DSSS modulation.

TABLE 1

Probe connection failure rates

| Subject | Probe Connection Failure | |
|---|---|---|
| | WB-DSSS | GFSK |
| 1 | 8.64% | 4.76% |
| 2 | 22.22% | |
| 3 | 13.76% | |
| 4 | 49.41% | 11.11% |
| 5 | 0.93% | 0.00% |
| 6 | 25.11% | |
| 7 | 8.65% | 3.70% |
| 8 | 22.22% | 0.00% |
| 9 | 3.80% | |
| 10 | 15.31% | |
| 11 | 13.25% | 0.00% |
| 12 | 17.39% | |
| 13 | 3.66% | |
| 14 | 3.68% | |
| 15 | 38.46% | |
| 16 | | 0.00% |
| 17 | | 0.00% |
| Average Failure Rate | 14.65% | 2.17% |

Overall, these data show that both the WB-DSSS and GFSK modulation schemes performed better than a system without the relay device, which was not able to create a connection between the device and a smartphone.

### Example 3. Treatment of Urinary Incontinence Utilizing a Relay Device with an Intravaginal Device

An intravaginal device (2) that includes a plurality of accelerometers positioned along a length of the device may be used to treat a subject having urinary incontinence (UI). The subject may have been identified as having a risk for developing UI (e.g., a subject who has recently experienced vaginal childbirth) or have been diagnosed as having UI by a medical practitioner. Alternatively, the subject experiencing the symptoms of UI may self-identify as having a need to train her pelvic floor muscles to reduce the frequency and/or the severity of UI symptoms. The subject may obtain the device from a medical practitioner or from a retail outlet.

The intravaginal device (2) may be an elongate probe that comes with a cover sheath. The cover sheath may contain an RF transceiver relay device (8) as described herein. The subject has a personal smartphone device (6). The subject downloads an application that is configured to run in conjunction with the intravaginal device (2). The subject may begin by removing the cover sheath and placing it in her side pants pocket. The subject may insert the intravaginal device into her vagina and position it proximal to the cervix or, for a subject with a hysterectomy, to the vaginal cuff.

The subject will then perform a series of pelvic floor lifts (PFLs) to strengthen her pelvic floor muscles. The subject can perform a series of PFLs, e.g., for 2 minutes and then rest the muscles for 2 minutes, repeating the series for a total of 5 times over 20 minutes. The device measures and collects data via the accelerometers on the device. The position and movement data obtained from the sensors is transmitted to the radio frequency transceiver at 915 MHz, the ISM RF band. The transceiver (8) receives the signal from the accelerometers and converts the signal to 2.45 GHz, the BLE RF band. The transceiver (8) transmits the signal to the peripheral device (6). The peripheral device may process the data obtained from the transceiver, e.g., using an algorithm, in order to display real-time data on a graphical user interface of the smartphone (6). The graphical user interface may display data such as strength of the activation of the pelvic floor muscles during the pelvic floor exercise and duration of time during which activation is maintained. The subject will perform this training program at least once a day (or more, e.g., three times per day) for about one week to about three months. The intravaginal device (2) may be removed before each set of exercises or the device may remain inserted for the duration of the treatment period. The smartphone application may track and score this data over time to provide the subject feedback as to the improvement of her pelvic floor muscle strength and symptoms. Over time, the symptoms resolve.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A radio frequency (RF) transceiver configured to transmit and receive a first RF signal at a first frequency of greater than about 1 GHz and a second RF signal at a second frequency of less than about 1 GHz.

2. The transceiver of claim 1, wherein the first frequency is greater than about 2 GHz.

3. The transceiver of claim 2, wherein the first frequency is from about 2 GHz to about 3 GHz.

4. The transceiver of claim 3, wherein the first frequency is about 2.45 GHz.

5. The transceiver of any one of claims 1-4, wherein the second frequency is from about 1 MHz to about 1 GHz.

6. The transceiver of claim 5, wherein the second frequency is about 915 MHz, about 433 MHz, or about 402 MHz.

7. The transceiver of any one of claims 1-6, wherein the transceiver is configured to transmit to and to receive an RF signal from a Bluetooth Low Energy (BLE) device that is configured to transmit and/or receive the first RF signal at the first frequency, wherein, optionally, the BLE device is configured to transmit and/or receive the first RF signal at the first frequency.

8. The transceiver of any one of claims 1-7, wherein the transceiver is configured to transmit to and to receive the second RF signal at the second frequency from an industrial, scientific, and medical (ISM) or Medical Implant Communications Systems (MICS) device, wherein, optionally, the ISM or MICS device is configured to transmit and/or receive the second RF signal at the second frequency.

9. The transceiver of any one of claims 1-8, further comprising a microcontroller.

10. A system comprising:
(a) a relay device comprising the transceiver of any one of claims 1-9;
(b) a first device comprising a first transmitter and/or receiver configured to transmit and/or receive the first RF signal at the first frequency; and
(c) a second device comprising a second transmitter and/or receiver configured to transmit and/or receive the second RF signal at the second frequency.

11. The system of claim 10, wherein the first device is a BLE device.

12. The system of claim 10 or 11, wherein the second device is an ISM or MICS RF device.

13. The system of any one of claims 10-12, further comprising a peripheral device.

14. The system of claim 13, wherein the peripheral device comprises the first device and/or the relay device.

15. The system of claim 13 or 14, wherein the peripheral device is a smartphone, tablet, computer, or smart watch.

16. The system of any one of claims 10-15, further comprising a medical device.

17. The system of claim 16, wherein the medical device comprises the second device.

18. The system of any one of claims 10-17, further comprising one or more microcontrollers.

19. The system of claim 18, wherein the one or more microcontrollers are in the medical device and/or the peripheral device.

20. The system of any one of claims 16-19, wherein at least a portion of the medical device is configured to be inserted or implanted inside a body or body cavity of a subject.

21. The system of claim 20, wherein the portion of the medical device comprises the second device.

22. The system of claim 20 or 21, wherein the subject is a human.

23. The system of any one of claims 16-22, wherein the medical device is an intravaginal, intrarectal, or intraurethral device.

24. The system of any one of claims 16-23, wherein the medical device comprises at least one sensor that is connected to the second device, wherein, optionally, the connection is a wired or wireless connection.

25. The system of claim 24, wherein the at least one sensor is configured to transmit a signal to the second device.

26. The system of claim 24 or 25, wherein the medical device comprises a plurality of sensors.

27. The system of claim 26, wherein medical device comprises a plurality of sensors positioned along a length of the medical device.

28. The system of any one of claims 24-27, wherein the at least one sensor is a position or movement sensor.

29. The system of claim 28, wherein the position or movement sensor is a microelectromechanical (MEM) sensor.

30. The system of claim 28 or 29, wherein the position or movement sensor is an accelerometer.

31. The system of any one of claims 16-30, wherein the medical device further comprises at least one sensor selected from the group consisting of a pressure sensor, a flow sensor, a muscle quality sensor, a muscle strength sensor, a pH sensor, a humidity sensor, a temperature sensor, a hormone sensor, a toxin sensor, and a hall effect sensor.

32. The system of claim 31, wherein the medical device comprises the hall effect sensor.

33. A method of using the system of any one of claims 10-32 comprising:
(a) transmitting the second RF signal at the second frequency from the second device to the relay device;
(b) receiving the second RF signal at the second frequency by the relay device;
(c) converting the second RF signal at the second frequency by the relay device to the first RF signal at the first frequency; and
(d) transmitting the first RF signal at the first frequency to the first device.

34. A method of using the system of any one of claims 10-32 comprising:
(a) transmitting the first RF signal at the first frequency from the first device to the relay device;
(b) receiving the first RF signal at the first frequency by the relay device;
(c) converting the first RF signal at the first frequency by the relay device to the second RF signal at the second frequency; and
(d) transmitting the second RF signal at the second frequency to the second device.

35. A method of detecting a pelvic floor movement with the system of any one of claims 10-32 comprising:
(a) inserting a medical device comprising the second device into the body of the subject, wherein the medical device is an intravaginal, intrarectal, or intraurethral device comprising at least one position or movement sensor;
(b) obtaining a signal from the position or movement sensor upon engagement or contraction of the pelvic floor; and
(c) transmitting the signal from the position or movement sensor to a peripheral device comprising the first device via the transceiver.

36. The method of claim 34 or 35, wherein the first frequency is greater than about 2 GHz.

37. The method of claim 36, wherein the first frequency is from about 2 GHz to about 3 GHz.

38. The method of claim 37, wherein the first frequency is about 2.45 GHz.

39. The method of any one of claims 34 to 38, wherein the second frequency is from about 1 MHz to about 1 GHz.

40. The method of claim 39, wherein the second frequency is about 915 MHz, about 433 MHz, or about 402 MHz.

41. The method of any one of claims 38 to 40, wherein the first frequency is about 2.45 GHz and the second RF frequency is about 915 MHz.

42. The method of claim 35, wherein the intravaginal, intrarectal, or intraurethral device comprises a plurality of position or movement sensors.

43. The method of claim 42, wherein the plurality of position or movement sensors are MEM sensors.

44. The method of claim 43, wherein the MEM sensors are accelerometers.

45. The method of claim 42 or 43, wherein the MEM sensors are positioned along a length of the device.

46. The method of any one of claims 43 to 45, wherein a position of the subject’s vagina, rectum, or urethra that is generated by the plurality of position or movement sensors is displayed on a graphical user interface of the peripheral device.

47. The method of claim 46, wherein the position of the subject’s vagina, rectum, or urethra is displayed on the graphical user interface prior to performance of a pelvic floor exercise and/or after performance of the pelvic floor exercise.

48. The method of claim 47, wherein the position of the subject’s vagina, rectum, or urethra prior to performance of a pelvic floor exercise and/or after performance of the pelvic floor exercise is recorded.

49. A medical device comprising the transceiver of any one of claims 1-9.

50. The medical device of claim 49, wherein the medical device is an intravaginal, intrarectal, or intraurethral device.

51. The medical device of claim 49 or 50, wherein the medical device comprises a plurality of position or movement sensors.

52. The medical device of claim 51, wherein the plurality of position or movement sensors are MEM sensors.

53. The medical device of claim 52, wherein the MEM sensors are accelerometers.

54. The medical device of claim 52 or 53, wherein the MEM sensors are positioned along a length of the device.

55. A kit comprising
(a) a relay device comprising the transceiver of any one of claims 1-9;
(b) a first device comprising a first transmitter and/or receiver configured to transmit and/or receive the first RF signal at the first frequency; and/or
(c) a second device comprising a second transmitter and/or receiver configured to transmit and/or receive the second RF signal at the second frequency.

56. The kit of claim 55, further comprising a medical device.

57. The kit of claim 56, wherein the medical device is an intravaginal, intrarectal, or intraurethral device.

58. The kit of claim 55 or 56, wherein the medical device comprises a plurality of position or movement sensors.

59. The kit of claim 58, wherein the plurality of position or movement sensors are MEM sensors.

60. The kit of claim 59, wherein the MEM sensors are accelerometers.

61. The kit of any one of claims 55 to 60, wherein the medical device comprises the second device.

62. The kit of any one of claims 55 to 61, further comprising instructions for use thereof.

63. The transceiver of claim 1, wherein the second frequency is from about 1 MHz to about 1 GHz.

64. The transceiver of claim 63, wherein the second frequency is about 915 MHz, about 433 MHz, or about 402 MHz.

65. The transceiver of claim 1, wherein the transceiver is configured to transmit to and to receive an RF signal from a Bluetooth Low Energy (BLE) device that is configured to transmit and/or receive the first RF signal at the first frequency, wherein, optionally, the BLE device is configured to transmit and/or receive the first RF signal at the first frequency.

66. The transceiver of claim 1, wherein the transceiver is configured to transmit to and to receive the second RF signal at the second frequency from an industrial, scientific, and medical (ISM) or Medical Implant Communications Systems (MICS) device, wherein, optionally, the ISM or MICS device is configured to transmit and/or receive the second RF signal at the second frequency.

67. The transceiver of claim 1, further comprising a microcontroller.

68. A system comprising:
(a) a relay device comprising the transceiver of claim 1;
(b) a first device comprising a first transmitter and/or receiver configured to transmit and/or receive the first RF signal at the first frequency; and
(c) a second device comprising a second transmitter and/or receiver configured to transmit and/or receive the second RF signal at the second frequency.

69. The system of claim 68, wherein the first device is a BLE device.

70. The system of claim 68, wherein the second device is an ISM or MICS RF device.

71. The system of claim 68, further comprising a peripheral device.

72. The system of claim 71, wherein the peripheral device comprises the first device and/or the relay device.

73. The system of claim 71, wherein the peripheral device is a smartphone, tablet, computer, or smart watch.

74. The system of claim 68, further comprising a medical device.

75. The system of claim 74, wherein the medical device comprises the second device.

76. The system of claim 68, further comprising one or more microcontrollers.

77. The system of claim 76, wherein the one or more microcontrollers are in the medical device and/or the peripheral device.

78. The system of claim 74, wherein at least a portion of the medical device is configured to be inserted or implanted inside a body or body cavity of a subject.

79. The system of claim 78, wherein the portion of the medical device comprises the second device.

80. The system of claim 78, wherein the subject is a human.

81. The system of claim 74, wherein the medical device is an intravaginal, intrarectal, or intraurethral device.

82. The system of claim 74, wherein the medical device comprises at least one sensor that is connected to the second device, wherein, optionally, the connection is a wired or wireless connection.

83. The system of claim 82, wherein the at least one sensor is configured to transmit a signal to the second device.

84. The system of claim 82, wherein the medical device comprises a plurality of sensors.

85. The system of claim 84, wherein medical device comprises a plurality of sensors positioned along a length of the medical device.

86. The system of claim 82, wherein the at least one sensor is a position or movement sensor.

87. The system of claim 86, wherein the position or movement sensor is a microelectromechanical (MEM) sensor.

88. The system of claim 86, wherein the position or movement sensor is an accelerometer.

89. The system of claim 74, wherein the medical device further comprises at least one sensor selected from the group consisting of a pressure sensor, a flow sensor, a muscle quality sensor, a muscle strength sensor, a pH sensor, a humidity sensor, a temperature sensor, a hormone sensor, a toxin sensor, and a hall effect sensor.

90. The system of claim 89, wherein the medical device comprises the hall effect sensor.

91. A method of using the system of claim 68 comprising:
(a) transmitting the second RF signal at the second frequency from the second device to the relay device;
(b) receiving the second RF signal at the second frequency by the relay device;
(c) converting the second RF signal at the second frequency by the relay device to the first RF signal at the first frequency; and
(d) transmitting the first RF signal at the first frequency to the first device.

92. A method of using the system of claim 68 comprising:
(a) transmitting the first RF signal at the first frequency from the first device to the relay device;
(b) receiving the first RF signal at the first frequency by the relay device;
(c) converting the first RF signal at the first frequency by the relay device to the second RF signal at the second frequency; and
(d) transmitting the second RF signal at the second frequency to the second device.

93. A method of detecting a pelvic floor movement with the system of claim 68 comprising:
(a) inserting a medical device comprising the second device into the body of the subject, wherein the medical device is an intravaginal, intrarectal, or intraurethral device comprising at least one position or movement sensor;
(b) obtaining a signal from the position or movement sensor upon engagement or contraction of the pelvic floor; and
(c) transmitting the signal from the position or movement sensor to a peripheral device comprising the first device via the transceiver.

94. The method of claim 92, wherein the first frequency is greater than about 2 GHz.

95. The method of claim 94, wherein the first frequency is from about 2 GHz to about 3 GHz.

96. The method of claim 95, wherein the first frequency is about 2.45 GHz.

97. The method of claim 92, wherein the second frequency is from about 1 MHz to about 1 GHz.

98. The method of claim 97, wherein the second frequency is about 915 MHz, about 433 MHz, or about 402 MHz.

99. The method of claim 96, wherein the first frequency is about 2.45 GHz and the second RF frequency is about 915 MHz.

100. The method of claim 93, wherein the intravaginal, intrarectal, or intraurethral device comprises a plurality of position or movement sensors.

101. The method of claim 100, wherein the plurality of position or movement sensors are MEM sensors.

102. The method of claim 101, wherein the MEM sensors are accelerometers.

103. The method of claim 101, wherein the MEM sensors are positioned along a length of the device.

104. The method of claim 101, wherein a position of the subject’s vagina, rectum, or urethra that is generated by the plurality of position or movement sensors is displayed on a graphical user interface of the peripheral device.

105. The method of claim 104, wherein the position of the subject’s vagina, rectum, or urethra is displayed on the graphical user interface prior to performance of a pelvic floor exercise and/or after performance of the pelvic floor exercise.

106. The method of claim 105, wherein the position of the subject’s vagina, rectum, or urethra prior to performance of a pelvic floor exercise and/or after performance of the pelvic floor exercise is recorded.

107. A medical device comprising the transceiver of claim 1.

108. The medical device of claim 107, wherein the medical device is an intravaginal, intrarectal, or intraurethral device.

109. The medical device of claim 107, wherein the medical device comprises a plurality of position or movement sensors.

110. The medical device of claim 109, wherein the plurality of position or movement sensors are MEM sensors.

111. The medical device of claim 110, wherein the MEM sensors are accelerometers.

112. The medical device of claim 110, wherein the MEM sensors are positioned along a length of the device.

113. A kit comprising
(a) a relay device comprising the transceiver of claim 1;
(b) a first device comprising a first transmitter and/or receiver configured to transmit and/or receive the first RF signal at the first frequency; and/or
(c) a second device comprising a second transmitter and/or receiver configured to transmit and/or receive the second RF signal at the second frequency.

114. The kit of claim 113, further comprising a medical device.

115. The kit of claim 113, wherein the medical device is an intravaginal, intrarectal, or intraurethral device.

116. The kit of claim 113, wherein the medical device comprises a plurality of position or movement sensors.

117. The kit of claim 116, wherein the plurality of position or movement sensors are MEM sensors.

118. The kit of claim 117, wherein the MEM sensors are accelerometers.

119. The kit of claim 113, wherein the medical device comprises the second device.

120. The kit of claim 113, further comprising instructions for use thereof.
